(19) United States
(12) Patent Application Publication
(10) Pub. No.: US 2023/0256153 A1
(43) Pub. Date: Aug. 17, 2023

(54) EXTREMITY IRRIGATION DEBRIDEMENT BASIN WITH PRESSURIZED FLUID

(71) Applicant: Pedro Juan LOREDO, Southlake, TX (US)

(72) Inventor: Pedro Juan LOREDO, Southlake, TX (US)

(21) Appl. No.: 17/671,662

(22) Filed: Feb. 15, 2022

(51) Int. Cl. A61M 1/00

(52) U.S. Cl. CPC A61M 1/60; A61M 1/84; A61M 1/77; A61M 2210/083; A61M 2210/086

(57) ABSTRACT

An extremity irrigation debridement basin includes a body having a floor and a sidewall extending between the floor and an upper rim. An opening is positioned opposite the floor and defined by the upper rim. A shield extends upward from the upper rim of the sidewall. A recess is formed in the sidewall. The recess extends from an upper rim opening toward the floor.

### CROSS-REFERENCE TO RELATED APPLICATIONS

This application claims priority under 35 U.S.C. § 119(e) to U.S. Provisional Application No. 63/286,212, filed Dec. 6, 2021, which is expressly incorporated by reference herein.

### TECHNICAL FIELD

The present invention is directed to basins for use in medical, hospital, healthcare, nursing, and other facilities, and in particular, a portable basin having an extremity recess and splash shield.

### BACKGROUND

Portable basins are typically is used in emergency rooms, trauma centers, surgical suites, and operating rooms for wound irrigation on persons who generally are immobilized. Most portable basins are constructed from a hard plastic, are generally in a bowl or box shape, and include a narrow rounded upper rim. In use, a user places an appendage of a subject over the upper rim of the basin and irrigates an area of the appendage with fluids. The fluids drain off the appendage and into the basin. Any flesh, bone or debris likewise is collected in the bottom of the basin.

Many irrigation basins are of a rounded bowl or box shaped configuration. When such basins are used adjacent other appendages, the adjacent appendage must be re-positioned, many times in an awkward position to accommodate the surgical basin. This can be uncomfortable for the patient, and it can be difficult for the healthcare provider to maintain the positioning.

### SUMMARY

The present disclosure includes one or more of the features recited in the appended claims and/or the following features which, alone or in any combination, may comprise patentable subject matter.

According to a first aspect of the disclosed embodiments, an extremity irrigation debridement basin includes a body having a floor and a sidewall extending between the floor and an upper rim. The floor and the sidewall define a chamber. An opening is positioned opposite the floor and is defined by the upper rim. A shield extends upward from the upper rim of the sidewall. A recess is formed in the sidewall. The recess extends from an upper rim opening toward the floor. At least one irrigation device extends through the sidewall. The at least one irrigation device is configured to dispense fluid into the chamber.

In some embodiments of the first aspect, a plurality of irrigation devices may be positioned around the sidewall. Each of the plurality of irrigation devices may dispense the fluid toward a center of the chamber. The at least one irrigation device may be coupled to a source of the fluid. The source of the fluid may be pressurized. At least one port may extend through the sidewall and may be positioned adjacent the floor of the body. The at least one port may be positioned above the floor of the body and below a bottom edge of the recess. The at least one port may be configured to couple to a suction device to suction the fluid from the chamber. The body may include a first end and a second end. The at least one irrigation device may be positioned adjacent the first end. The at least one port may be positioned adjacent the second end. The shield may extend from the upper rim at the first end. The recess may be formed in the second end. The shield may extend partially over the opening of the body from the first end toward the second end. The recess may extend through the sidewall to a bottom edge formed in the sidewall and may be positioned between the upper rim and the floor of the body.

According to a second aspect of the disclosed embodiments, an extremity irrigation debridement basin includes a body including a first end and a second end. The body has a floor and an upper rim extending between the first end and the second end. A sidewall extends between the floor and the upper rim. The floor, the sidewall, and the upper rim define a chamber. An opening is positioned opposite the floor and is defined by the upper rim. A recess is formed in the sidewall. The recess extends from an upper rim opening toward the floor. An irrigation device extends through the sidewall at the first end of the body. The irrigation device is configured to dispense fluid into the chamber. A port extends through the sidewall at the second end of the body. The port is configured to discharge the fluid from the chamber.

In some embodiments of the second aspect, the irrigation device may dispense the fluid toward a center of the chamber. The irrigation device may be coupled to a source of the fluid. The source of the fluid may be pressurized. The port may be positioned adjacent the floor of the body. The port may be positioned above the floor of the body and below a bottom edge of the recess. The port may be configured to couple to a suction device to suction the fluid from the chamber. A shield may extend partially over the opening of the body from the first end toward the second end. The recess may be formed in the second end.

### BRIEF DESCRIPTION OF THE DRAWINGS

The detailed description particularly refers to the accompanying figures in which:

FIG. 1 is a top perspective view of an extremity irrigation debridement basin in accordance with an embodiment;

FIG. 2 is a side elevation view of the basin shown in FIG. 1;

FIG. 3 is a first end perspective view of the basin shown in FIG. 1;

FIG. 4 is expanded view of a port of the basin shown in FIG. 1;

FIG. 5 is an elevation view of an alternative embodiment of a recess of the basin; and

FIG. 6 is a front view of a source of fluid in the form of a saline bag coupled to the pump shown in FIG. 3.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

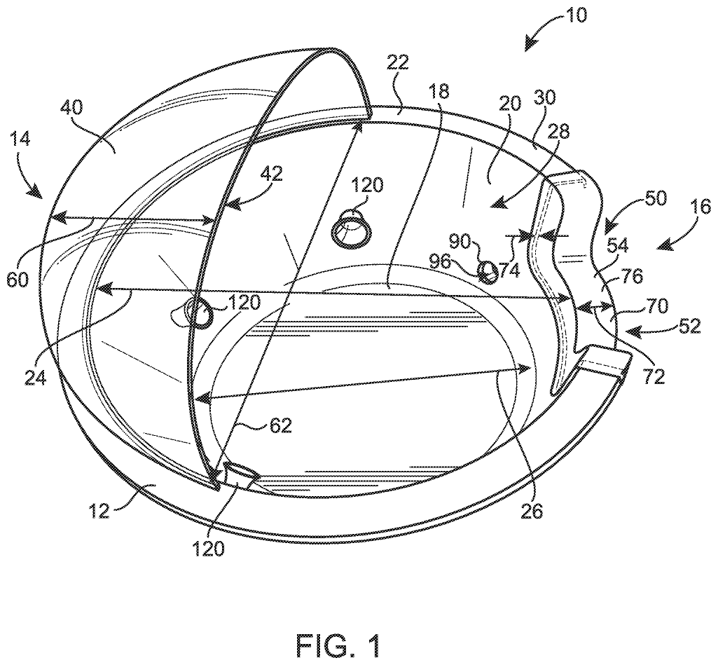

Referring to FIG. 1, an extremity irrigation debridement basin 10 is configured for irrigation of a patient's extremity during a surgical procedure. For example, during the operation of a patient's extremity, e.g. hand and/or foot, the caregiver may requiring flushing of blood and debris, e.g. bone, from the extremity. The basin 10 is configured so that the extremity is positioned within the basin 10 with the patient's arm or leg resting within a recess (described in detail below) of the basin 10. The basin 10 is configured to collect fluid and debris from the extremity. A suction device may be coupled to the basin 10, as described below, to suction the fluid and debris from the basin 10, during and after irrigation of the extremity.

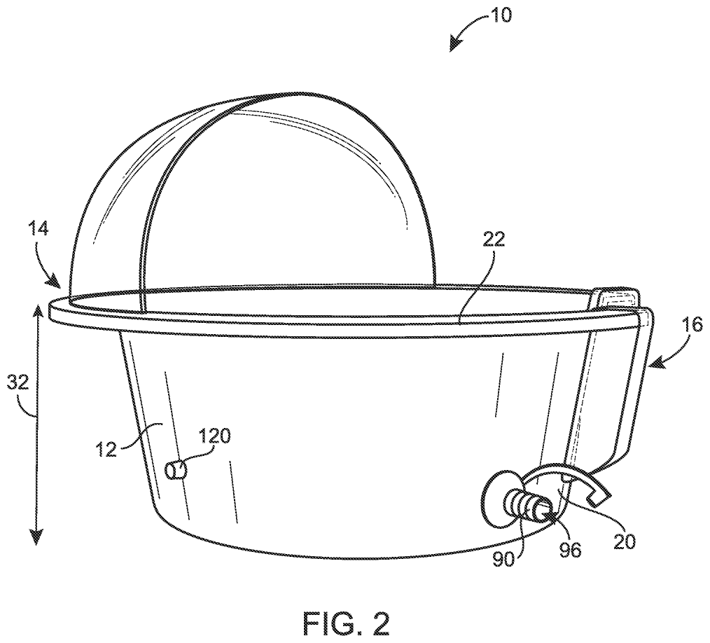

The basin 10 includes a body 12 having a first end 14 and an opposite second end 16. The basin 10 includes a floor 18 extending between the first end 14 and the second end 16. A sidewall 20 extends upward from the floor 18 to an upper rim 22. In the exemplary embodiment, the floor 18 is circular and the upper rim 22 is circular. It will be appreciated that other shapes for the floor 18 and the upper rim 22 may be contemplated. The sidewall 20 extends between the floor 18 and the upper rim 22 at an oblique angle so that the upper rim 22 has a larger diameter 24 than a diameter 26 of the floor 18. As shown in FIG. 2, the sidewall 20 has a height 32. In an exemplary embodiment, the height 32 is approximately 4.75 inches; however the sidewall 20 may have any suitable height 32. In an exemplary embodiment, the diameter 24 is approximately 13.5 inches; however, the diameter 24 may be any suitable length. An opening 28 is formed at a top 30 of the basin 10. The opening 28 is defined by the upper rim 22. Generally, the floor 18, the sidewall 20, the upper rim 22, and the opening 28 form a bowl configuration. The floor 18, the sidewall 20, and the upper rim 22 define a chamber 30 within the body 12. The chamber 30 includes a center 32.

A splash shield 40 is positioned at the first end 14. In some embodiments, the shield 40 may be removable. The shield 40 is configured to prevent the discharge of fluid from the basin 10 when a patient's extremity is cleansed in the basin 10. In some embodiments, the shield 40 is rotatable around the upper rim 22 to position the shield 40 for preventing the discharge of fluid. The shield 40 is formed from a transparent material that enables visibility of the basin 10 during a cleansing procedure. The shield 40 extends upward from the upper rim 22. The shield 40 extends a length 62. In an exemplary embodiment, the length 62 may be 12 inches; however, the shield 40 may have any suitable length 62. The shield 40 also extends partially from the first end 14 toward the second end 16 over the opening 28 at a length 60. In an exemplary embodiment, the length 60 is approximately 6 inches; however the shield 40 may extend any suitable length 60. The shield 40 is curved and generally arcuate to from a space 42 between the shield 40 and the basin 10. The space 42 provides room for a caregiver to cleanse the patient's extremity when the patient's extremity is positioned in the basin 10.

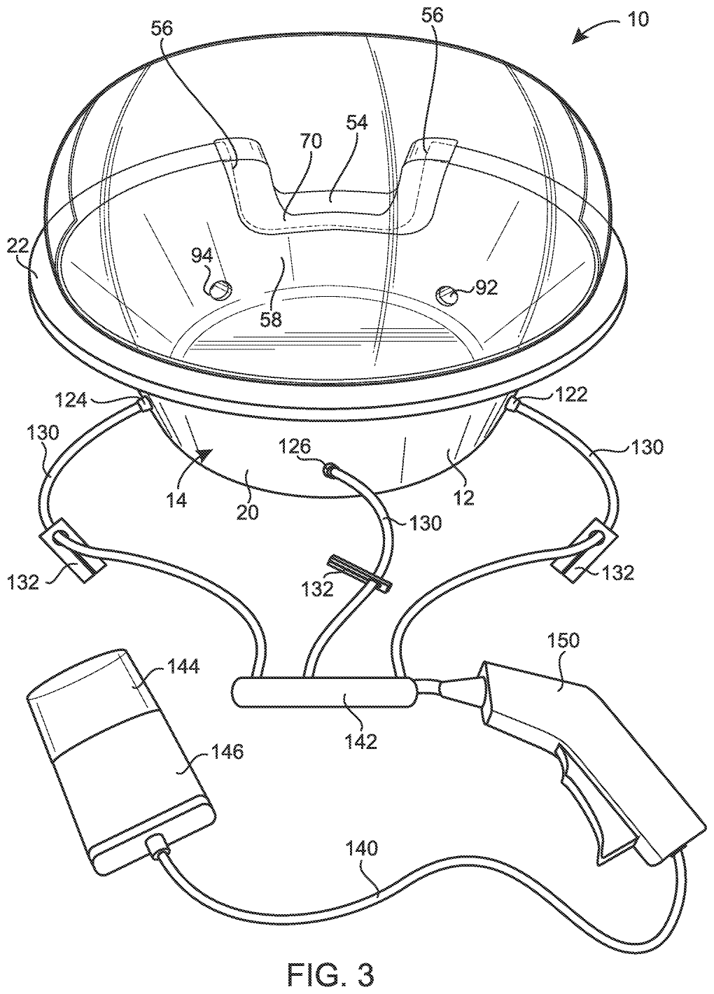

A recess 50 is formed in the second end 16. The recess 50 extends from an upper rim opening 52 in the upper rim 22 to a bottom edge 54. A pair of side edges 56 (shown in detail in FIG. 3) extend between the upper rim 22 and the bottom edge 54. The recess 50 is defined by the side edges 56 and the bottom edge 54. The recess 50 extends partially through the sidewall 20 from the upper rim 22 toward the floor 18. The bottom edge 54 is positioned between the upper rim 22 and the floor 18 of the basin 10. Accordingly, a sidewall portion 58 extends between the bottom edge 54 and the floor 18 to prevent fluids from spilling through the recess 50. The recess 50 is configured to receive an extremity of the patient.

A ledge 70 extends across the bottom edge 54. The ledge 70 has a width 72 that is greater than a thickness 74 of the sidewall 20. As shown, in FIG. 2, the ledge 70 extends outward from the sidewall 20. As shown in FIG. 3, the ledge also extends inward from the sidewall 20 into the basin 10. The ledge 70 has a smooth, flat surface 76. In some embodiments, the ledge 70 may include a smooth, curved surface that generally conforms with the contours of a patient's extremities. When the patient's extremity is positioned in the basin a portion of the patient's extremity is positioned on the ledge 70 for comfort. For example, when a patient's hand is positioned in the basin 10, the patient's arm is positioned on the ledge 70. In another example, when a patient's foot is positioned in the basin 10, the patient's leg is positioned on the ledge 70.

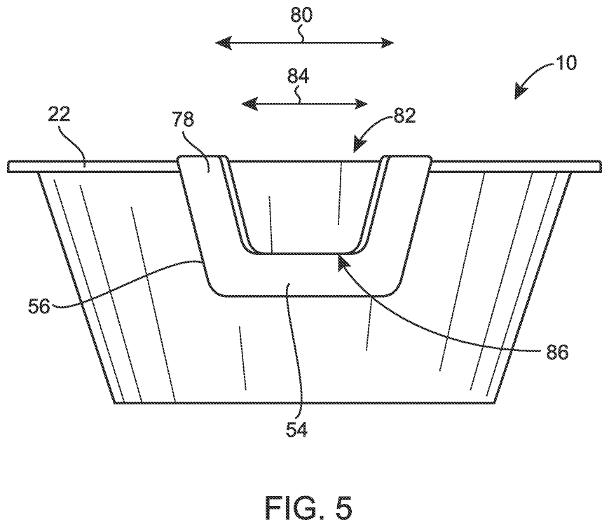

As shown in FIG. 5, the recess 50 has a length 80 at the top 82, which is greater than a length 84 at the bottom 86. In an exemplary embodiment, the length 80 is approximately 6 inches, and the length 84 is approximately 2.5 inches; but the recess 50 can have any suitable length 80 and length 84. In some embodiments, as shown in FIG. 5, a flexible, water-proof, barrier 78 seals the recess 50. The barrier 78 is attached at the upper rim 22, the side edges 56, and the bottom edge 54. Accordingly, the barrier 78 fills at least a portion of the recess 50. The barrier 78 adaptably forms around and seals around an extremity to provide for a higher volume of fluid to be contained by the basin 10.

As illustrated in FIGS. 1 and 2, at least one suction port 90 extends from the sidewall 20. The suction port 90 includes a channel 96 that extends through the sidewall 20 to enable a suction device (not shown) that is coupled to the suction port to remove fluids and debris from the basin 10. The suction port 90 is position above the floor 18 of the basin 10 and below the bottom edge 54 of the recess 50. In the exemplary embodiment, the suction port 90 is positioned adjacent the floor 18 of the basin 10.

Referring to FIG. 3, the at least one suction port 90 includes a right side port 92 and a left side port 94. The ports 92 and 94 provide flexibility for the caregiver when coupling a suction device to the basin 10. That is, depending on the position of the basin 10 in a healthcare facility room, only one of the ports 92 or 94 may be accessible. In some embodiments, a suction device may be coupled to both suction ports 92 and 94. It will also be appreciated that suction ports 90 may be provided at other locations of the basin 10. For example, a suction port 90 may be positioned at the first end 14 or the second end 16 of the basin 10.

As illustrated in FIGS. 1-3, at least one irrigation device 120 extends through the sidewall 20 to dispense fluid into the chamber 30. In an exemplary embodiment, the irrigation device 120 is a jet or nozzle configured to eject pressurized fluid toward the center 32 of the chamber 30. As shown in FIG. 3, a plurality of irrigation devices 120 are positioned around the sidewall 20 and includes a right irrigation device 122, a left irrigation device 124, and a center irrigation device 126. Each of the right irrigation device 122, the left irrigation device 124, and the center irrigation device 126 are arranged to dispense the fluid toward the center 32 of the chamber 30. The right irrigation device 122, the left irrigation device 124, and the center irrigation device 126 are positioned at the first end 14 of the body 12 opposite the ports 90, which are positioned in the second end 16 of the body 12.

In some embodiments, irrigation devices 120 may be positioned at either the first end 14 or the second end 16 of the body 12. Also, the ports 90 may be positioned at either the first end 14 or the second end 16 of the body 12. For example, the irrigation devices 120 and ports 90 may be alternated around the sidewall 20 at each of the first end 14 and the second end 16 of the body 12. The irrigation devices 120 may be positioned above the ports 90 to provide fluid to the patient's extremity, while the ports 90 drain the fluid from the chamber 30.

Referring to FIG. 3, each of the irrigation devices 120 is coupled to an individual device hose 130. Each individual device hose 130 includes a clamp 132 configured to alter the device hose 130 from an open position and a closed position. Accordingly, any one of the right irrigation device 122, the left irrigation device 124, and the center irrigation device 126 may be closed by closing the respective device hose 130 with the respective clamp 132. Any number of device hoses 130 may be closed with the respective clamp 132.

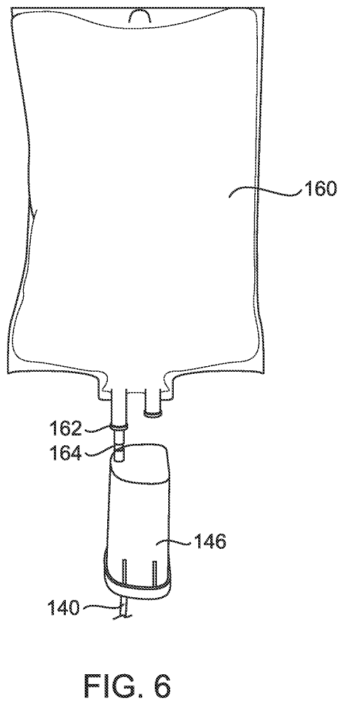

Each device hose 130 is connected to a main hose 140 via a hub 142. The main hose 140 is joined to a source of fluid 144. The fluid may be water or water mixed with medication and/or any other sanitizing fluid. The fluid may also be any medicated or sanitized fluid. The source of fluid 144 may be any source of fluid, for example, a faucet, a fluid bag, a fluid tank, or the like. A pump 146 may be provided in the main hose 140 to pressurize the fluid being discharged from the irrigation devices 120. In the embodiment illustrated in FIG. 6, the source of fluid 144 is a saline bag 160 having a port 162 that is coupled to a port 164 of the pump 146. In other embodiments, the fluid may be pressurized at the source of fluid 144. A triggering mechanism 150 may be provided in the main hose 140 downstream from the pump 146. The triggering mechanism 150 is configured to control the flow of the fluid through the main hose 140 to the irrigation devices 120. That is, in a resting position, the triggering mechanism 150 prevents the flow of the fluid from the main hose 140 to the device hoses 130. In an actuated position, the triggering mechanism 150 enables the flow of the fluid to the device hoses 130 so that the fluid can flow through the device hoses 130, that are in an open position, to the respective irrigation devices 120.

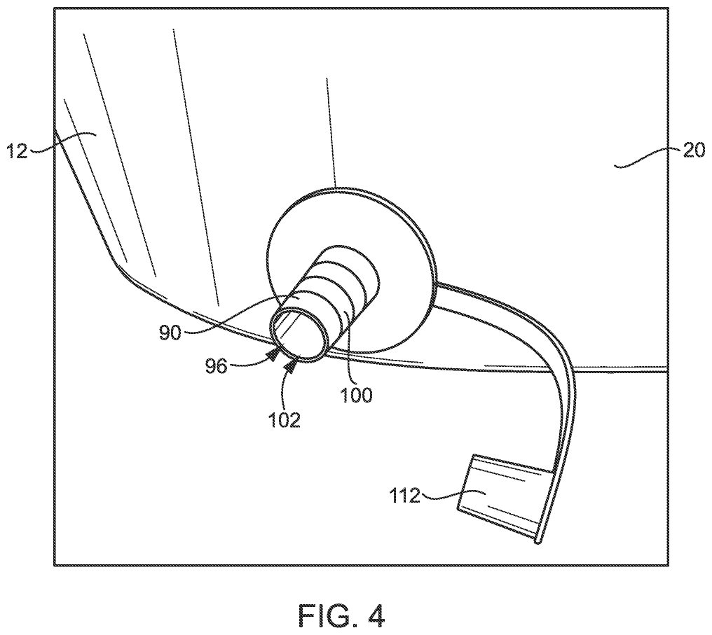

Referring now to FIG. 4, the suction port 90 includes a tube 100 that extends outward from the sidewall 20. The tube 100 provides access for a tube of the suction device to couple to the suction port 90. For example, the tube of the suction device may be inserted into the tube 100. Alternatively, the tube 100 may be inserted in the tube of the suction device. The channel 96 extends through the tube 100 from an opening 102 formed in an end 104 of the tube 100. A plug 112 is coupled to the tube 100 to seal the opening 102 when a suction device is not coupled to the tube 100.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. An extremity irrigation debridement basin comprising:
a body having a floor and a sidewall extending between the floor and an upper rim, wherein the floor and the sidewall define a chamber,
an opening opposite the floor and defined by the upper rim,
a shield extending upward from the upper rim of the sidewall,
a recess formed in the sidewall, wherein the recess extends from an upper rim opening toward the floor, and
a plurality of irrigation devices extending through the sidewall, wherein each of the plurality of irrigation devices is configured to dispense fluid into the chamber,
wherein each of the plurality of irrigation devices is coupled to one of a plurality of hoses and each of the plurality of hoses is coupled to a main hose that delivers the fluid to the plurality of irrigation devices from a source of fluid.

2. The basin of claim 1, wherein each of the plurality of irrigation devices is positioned around the sidewall.

3. The basin of claim 1, wherein each of the plurality of irrigation devices dispenses the fluid toward a center of the chamber.

4. (canceled)

5. The basin of claim 1, wherein the source of the fluid is pressurized.

6. The basin of claim 1, further comprising at least one port extending through the sidewall and positioned adjacent the floor of the body.

7. The basin of claim 6, wherein the at least one port is positioned above the floor of the body and below a bottom edge of the recess.

8. The basin of claim 6, wherein the at least one port is configured to couple to a suction device to suction the fluid from the chamber.

9. The basin of claim 6, wherein:
the body includes a first end and a second end,
at least one of the plurality of irrigation devices is positioned adjacent the first end, and
the at least one port is positioned adjacent the second end.

10. The basin of claim 9, wherein:
the shield extends from the upper rim at the first end, and
the recess is formed in the second end.

11. The basin of claim 10, wherein the shield extends partially over the opening of the body from the first end toward the second end.

12. The basin of claim 1, wherein the recess extends through the sidewall to a bottom edge formed in the sidewall and positioned between the upper rim and the floor of the body.

13. An extremity irrigation debridement basin comprising:
a body including a first end and a second end, the body having a floor and an upper rim extending between the first end and the second end,
a sidewall extending between the floor and the upper rim, wherein the floor, the sidewall, and the upper rim define a chamber,
an opening opposite the floor and defined by the upper rim,
a recess formed in the sidewall, wherein the recess extends from an upper rim opening toward the floor,
a plurality of irrigation devices extending through the sidewall at the first end of the body, wherein each of the plurality of irrigation devices is configured to dispense fluid into the chamber, wherein each of the plurality of irrigation devices is individually moveable between an open and closed position, and
a port extending through the sidewall at the second end of the body, wherein the port is configured to discharge the fluid from the chamber.

14. The basin of claim 13, wherein each of the plurality of irrigation devices dispenses the fluid toward a center of the chamber.

15. The basin of claim 13, wherein each of the plurality of irrigation devices is coupled to a source of the fluid.

16. The basin of claim 15, wherein the source of the fluid is pressurized.

17. The basin of claim 13, wherein the port is positioned adjacent the floor of the body.

18. The basin of claim 17, wherein the port is positioned above the floor of the body and below a bottom edge of the recess.

19. The basin of claim 13, wherein the port is configured to couple to a suction device to suction the fluid from the chamber.

20. The basin of claim 13, wherein:
a shield extends partially over the opening of the body from the first end toward the second end, and
the recess is formed in the second end.
